(19) United States
(12) Patent Application Publication
(10) Pub. No.: US 2024/0335294 A1
(43) Pub. Date: Oct. 10, 2024

(54) ORTHOPAEDIC DEVICE WITH CAM FOR A JOINT

(71) Applicant: EXOLAB, MARSEILLE (FR)

(72) Inventor: Gaetan AÜLLO-RASSER, SAINT-CYR-SUR-MER (FR)

(21) Appl. No.: 18/698,720

(22) PCT Filed: Oct. 12, 2022
(86) PCT No.: PCT/FR2022/051915

§ 371 Date: Apr. 4, 2024

(30) Foreign Application Priority Data
Oct. 25, 2021 (FR) 2111315

(51) Int. Cl. A61F 2/38

(52) U.S. Cl. CPC A61F 2/3845

(57) ABSTRACT

The present invention relates to an orthopedic device intended to equip a joint comprising an upper lever and a lower lever, the orthopedic device comprising an upper cam and a plate, the upper cam being joined to an upper fastening element provided to rigidly connect the upper cam to the upper lever, the plate being joined to a lower fastening element provided to rigidly connect the plate to the lower lever, characterized in that the upper cam has a cam surface whose generatrix has a convex curved profile, in the sagittal plane, and in that the plate and/or the upper cam are each connected to the associated fastening element by a single fastening rod.

### TECHNICAL FIELD OF THE INVENTION

The present invention relates to an orthopedic device intended to equip a joint, in particular a knee joint.

The technical field is that of joint prostheses and devices that require particular techniques due to the complexity of the joint, which must in particular keep its freedom of movement in a sagittal plane while ensuring the support of the weight of the body.

### TECHNICAL BACKGROUND

Joints such as the knee joint or the ankle joint are highly stressed. They can undergo damage in particular at the cartilage covering the femoral condyle and the tibial plate following a mechanical accident, due to wear and/or to the development of osteoarthritis. Various types of prostheses and devices have therefore been developed for many years to attempt to remedy such damage by replacing the natural surfaces damaged by artificial implants.

These devices, which aim to completely replace the knee joint, require surgery that is very serious for the patient. In addition, this type of operation is irreversible.

Alternatively, implantable prosthetic devices have been proposed, for example by document EP2585001B1, comprising at least two bearing parts not connected to one another, one being able to be attached on the tibia side and the other on the femur side. The two support parts are then positioned laterally and outwardly relative to the knee joint, with mobility relative to one another along a sagittal plane, on one side, for example the outside of the knee.

The system described and shown in document U.S. Pat. No. 4,637,382A1 is also known.

Tests carried out by the applicant have shown that it was possible to improve existing systems.

### SUMMARY OF THE INVENTION

The present invention aims to propose an improvement to known systems that are more easily implantable on the patient and easier to adapt to the patient, while being more economical to manufacture and more reliable. To this end, the invention proposes an orthopedic device intended to equip a joint, said joint comprising an upper lever and a lower lever, the respective ends of which have an upper bearing surface and a lower bearing surface opposite one another, said levers being pivotable relative to one another, around their respective bearing surfaces and in a pivoting plane called sagittal plane, between an extended position wherein said levers form between them an angle of at most 180 degrees, and a flexing position wherein said levers form between them an angle of at least 20 degrees, the orthopedic device comprising an upper cam and a plate not directly connected to one another, the upper cam being joined to an upper attachment element provided to rigidly connect the upper cam to the upper lever, the plate being joined to a lower attachment element provided to rigidly connect the plate to the lower lever, the upper cam and the plate being provided in order to be arranged at the outside of the joint so as to relieve the joint when it is under load, characterized in that the upper cam comprises a cam surface whose generatrix has a convex curved profile in the sagittal plane, in that the plate comprises a substantially flat upper track of a rectilinear profile in the sagittal plane, the profile of the cam surface and the relative positioning of the cam surface relative to the upper track being configured in order to ensure continuous contact of the upper cam against the upper track during the entire pivoting of the upper lever relative to the lower lever when the orthopedic device is mounted on the joint,

and in that the plate and/or the upper cam are each connected to the associated attachment element by a single fastening rod.

By virtue of the orthopedic device according to the invention, it is easier to adapt to the morphology of the patient since it is possible to retain a common attachment structure for several orthopedic devices. It is possible to modify only the profile of the cam surface from one patient to another, which makes it possible to manufacture standardized parts.

In addition, the solution according to the invention is simpler to implement than known devices. It is therefore less invasive and less complicated to implement while allowing optimal relief of the forces applied to the joint.

An advantage of the orthopedic device according to the invention is that it is a temporary implantable device. It can be worn by the patient for a determined duration, for example between two and three months. Thus, after disassembly and removal, no orthopedic material is left on the patient, in contrast to most current solutions.

After removal, the holes left by the fastening elements may optionally be filled, for example by using a bone filling biomaterial.

According to other features of the invention:
each fastening element consists of a pin which is provided to be attached in a portion of the associated lever of the joint, and the pin extends on the opposite side of the lever to form the fastening rod;
the fastening rod comprises a splined section and the upper cam and the plate are each provided with a splined hub element complementary to the associated splined section to allow the upper cam and the plate to be attached respectively to the associated fastening rod, the number of splines on the splined section determining the number of steps for the relative angular positioning of the upper cam and of the plate on the associated splined section;
the upper track is offset rearward relative to a vertical transverse plane passing through the associated fastening rod, when the orthopedic device is mounted on the joint;
the profile of the cam surface is configured so that the upper cam is bearing against the upper track by a front section of the cam surface when the orthopedic device is mounted on the joint and in the extended position, and so that the upper cam bears against the upper track by a rear section of the cam surface when the orthopedic device is mounted on the joint and in the flexing position;
the cam surface has several radii of curvature along its profile in the sagittal plane;
the main body forming the upper cam is of increasing transverse thickness from the fastening rod toward the cam surface;
the upper cam is produced in a single piece with the splined hub element;
the upper track is arranged on a shim that is removably attached to the plate;
the orthopedic device is configured to equip a knee joint, the upper lever corresponding to a femur and the lower lever corresponding to a tibia;

The invention also proposes a method for mounting an orthopedic device on a joint, the orthopedic device being produced according to any one of the preceding features, the joint comprising an upper lever and a lower lever, the respective ends of which have an upper bearing surface and a lower bearing surface opposite one another, said levers being pivotable relative to one another about their respective bearing surfaces and in a pivoting plane called sagittal plane, between an extended position wherein said levers form between them an angle of at most 180 degrees, and a flexing position wherein said levers form between them an angle of at least 20 degrees, the mounting method comprising the following steps:
determining the suitable location in the levers in order for attaching the pin-shaped fastening elements thereto,
making a single fastening hole in each lever,
attaching the fastening elements into their respective holes,
angularly positioning the plate on the associated fastening rod as a function of the angular position of the lower bearing surface when the joint is in its extended position,
attaching the plate to the associated fastening rod,
angularly positioning the upper cam on the associated fastening rod as a function of the angular position of the upper bearing surface when the joint occupies its extended position,
attaching the upper cam to the associated fastening rod.

According to an advantageous feature, the method further comprises a step of selecting the upper cam from a series of upper cams having cam surfaces with different profiles so as to approach the kinematics of the joint as closely as possible.

According to another advantageous feature, the method comprises a step of selecting a shim of a thickness determined as a function of the desired radial distance between the upper cam and the plate, and a step of fastening the shim to the plate.

### BRIEF DESCRIPTION OF THE FIGURES

Further features and advantages of the invention will become apparent from the following detailed description, which may be understood with reference to the attached drawings in which:

FIG. 1 is an exploded perspective view which schematically shows the orthopedic device according to the invention before assembly and mounting on a knee joint, the knee being shown schematically in the extended position;

FIG. 2 is a side view which schematically shows the orthopedic device of FIG. 1 when it is mounted on the knee joint in the extended position;

FIG. 3 is a view similar to that of FIG. 2 which depicts the orthopedic device in a first intermediate position where the knee joint is slightly flexed

FIG. 4 is a view similar to that of FIG. 2 which depicts the orthopedic device in a second intermediate position where the knee joint is more broadly flexed;

FIG. 5 is a view similar to that of FIG. 2 which shows the orthopedic device in a flexing position where the knee joint is flexed to about 90 degrees;

FIG. 6 is a perspective view which schematically shows the orthopedic device of FIG. 1 in the position that it occupies when it is mounted on the knee joint and the knee is in the extended position;

FIG. 7 is a front view which schematically shows the orthopedic device of FIG. 1 in the position shown in FIG. 6.

### DETAILED DESCRIPTION OF THE INVENTION

For the description of the invention and comprehension of the claims, the vertical, longitudinal and transverse orientations according to the reference frame V, L, T indicated in the figures, whose longitudinal L and transverse T axes extend in a horizontal plane, will be adopted in a non-limiting manner and without reference to earth's gravity.

By convention, the longitudinal axis L is oriented from the rear to the front.

In the following description, identical, similar or analogous elements will be referred to by the same reference numbers.

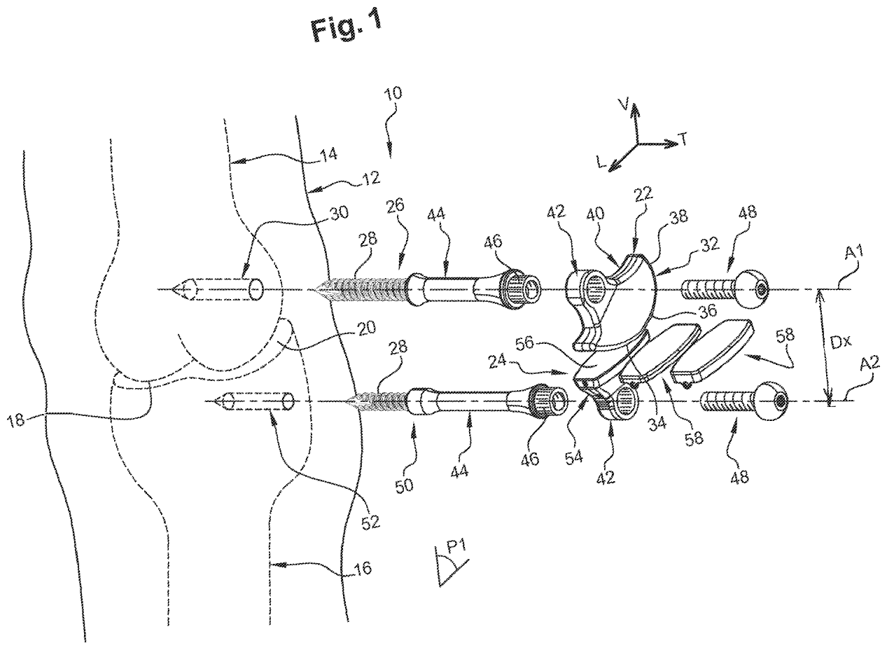

FIG. 1 shows an orthopedic device 10 intended to equip a knee joint 12, preferably on the outside of the joint 12. The knee extends generally in a vertical direction when its wearer is in the upright position, the joint 12 occupying a so-called extended position.

An orientation according to a sagittal plane P1 that extends in a vertical and longitudinal plane passing through the middle of the joint 12 will also be used.

The joint 12 thus comprises an upper lever 14, consisting here of the femur, and a lower lever 16, consisting here of the tibia. The end of the upper lever 14 comprises an upper bearing surface 18, or femoral condyle, and the end of the lower lever 16 comprises a lower bearing surface 20, or tibial plate.

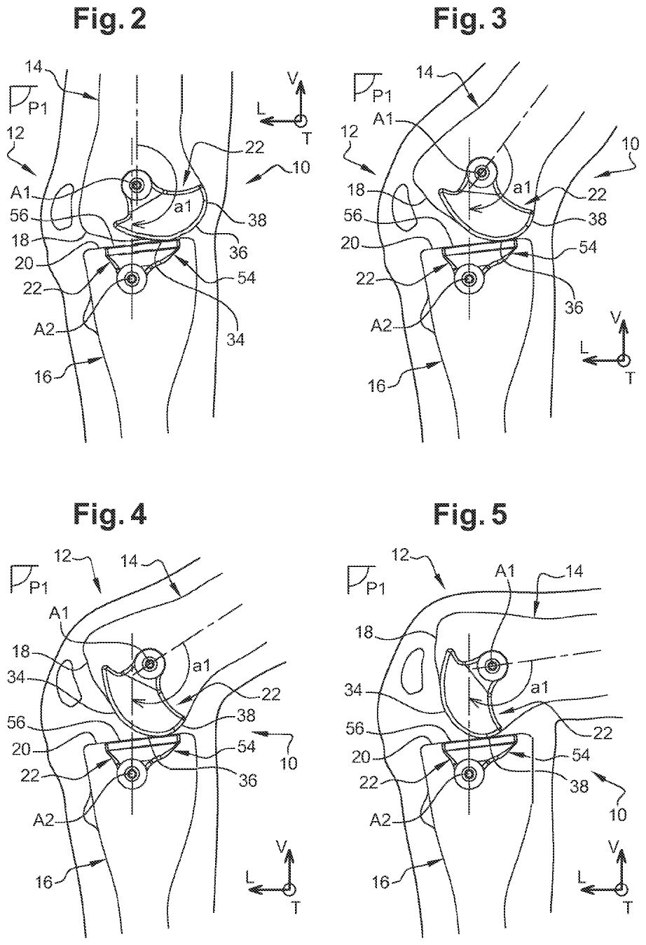

In the extended position, which is shown by FIGS. 1 and 2, the upper lever 14 describes with the lower lever 16 a flexing angle a1 substantially equal to 180 degrees.

FIGS. 2 to 5 show the different configurations of the joint 12 as a function of the angular positions taken by the upper lever 14 relative to the lower lever 16. From the extended position, which is shown by FIG. 2, the upper lever 14, which here corresponds to the femur, gradually pivots until reaching the flexing position shown by FIG. 5, wherein the upper lever 14 describes with the lower lever 16 a flexing angle a1 substantially equal to 90 degrees.

The pivoting of the upper lever 14 is mainly carried out in the sagittal plane P1, but there may be a slight angle setting of the upper lever 14 relative to the lower lever 16 in a horizontal plane.

During the pivoting of the upper lever 14, the upper bearing surface 18 is held bearing against the lower bearing surface 20, in particular under the effect of the patient's weight. The joint 12 is thus selectively subjected to a bearing force exerted on the respective bearing surfaces 18, 20 of the ends of said levers 14, 16.

The orthopedic device 10 according to an advantageous embodiment of the invention is now described in greater detail, in particular considering FIGS. 1, 6 and 7.

The orthopedic device 10 comprises an upper cam 22 and a plate 24 which are not connected directly to one another. The upper cam 22 and the plate 24 are provided to be arranged outside the joint 12 so as to provide additional or auxiliary bearing surfaces, which make it possible to relieve the joint 12 when it is under load. The orthopedic device 10 according to the invention makes it possible to relieve the bearing of the upper bearing surface 18 on the lower bearing surface 20, that is to say to relieve the bone-on-bone support.

The upper cam 22 is joined to an upper fastening element 26 which is provided to rigidly connect the upper cam 22 to the upper lever 14.

Advantageously, the upper fastening element 26 here consists of a pin comprising, on the side of the joint 12, a threaded end section 28 which allows the pin to be attached by screwing into an associated upper hole 30 produced beforehand in the upper lever 14.

The upper cam 22 comprises a cam surface 32 whose generatrix has a convex curved profile in the sagittal plane P1. The generatrix of the cam surface 32 is designed so as to form a profile that generally reproduces the curvature of the upper support surface 18, so that the pivoting and longitudinal movement of the upper cam 22 on the plate 24 reproduces the natural pivoting movement of the patient's knee. The upper cam 22 thus makes it possible to guide the pivoting of the joint 12.

Advantageously, the profile of the cam surface 32 is bio-inspired from the shape of the bones of the patient, that is to say that this profile is drawn so as to follow the pivoting profile of the femur on the tibial plate by relying on a modeled image from the joint 12 of the patient. In this case, the profile of the cam surface 32 is customized and specific to the patient.

Preferably, the profile of the cam surface 32 consists of several convex arcs of circle whose radii of curvature vary along the cam surface 32. Thus, in the example shown, the cam surface 32 has a front section 34 with a first radius of curvature r1, an intermediate section 36 with a second radius of curvature r2, and a rear section 38 with a third radius of curvature r3, where r1>r2>r3.

According to the embodiment shown here, the upper cam 22 comprises a main body 40, here generally in the shape of a half-cylinder, provided on the side opposite the cam surface 32 of a splined hub element 42 to allow its fastening to the upper fastening element 26.

Preferably, the transverse thickness of the main body 40 increases from the splined hub element 42 toward the cam surface 32, which allows the cam surface 32 to offer a bearing surface against the plate 24 sufficiently wide for good relief of the forces exerted on the joint 12, while minimizing the thickness of material necessary at the splined hub element 42.

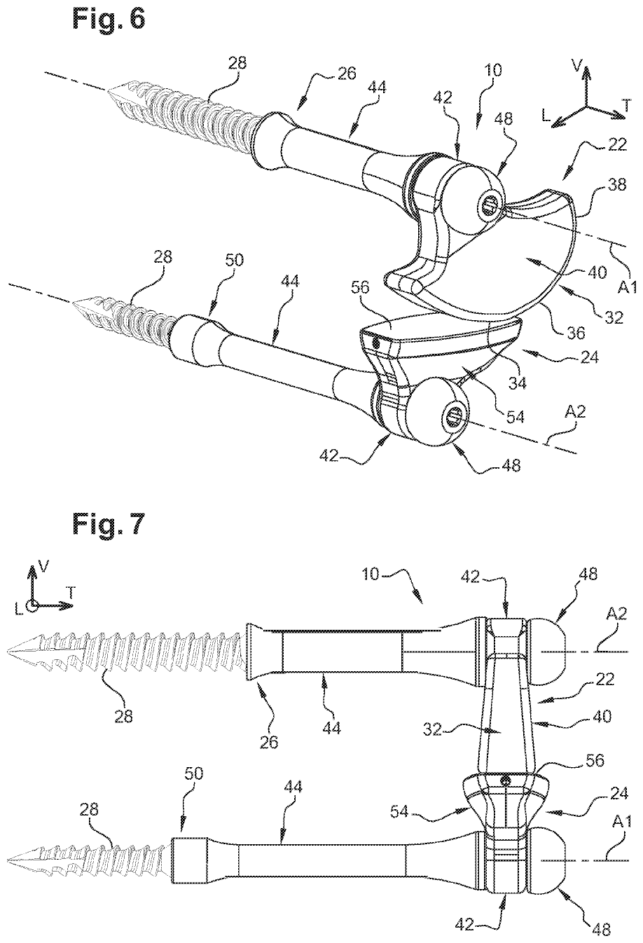

Advantageously, considering the extended position in FIG. 6, the main body 40 and the cam surface 32 are offset rearward relative to the splined hub element 42.

The upper cam 22 is connected to the upper fastening element 26 by a single fastening rod 44. The fastening rod 44 is here made in one piece with the upper fastening element 26, in the extension of the threaded end section 28. The fastening rod 44 comprises, at its free end, on the side opposite the joint 12, a splined section 46 complementary to the splined hub element 42 so as to allow the upper cam 22 to be engaged on the splined section 46. The upper cam 22 is thus pivotally joined to the upper fastening element 26 by a spline coupling which allows the upper cam 22, when it is attached to the upper lever 14, to pivot together with the upper lever 14 and with the upper bearing surface 18.

Preferably, a locking screw 48 is screwed into the splined section 46 so as to axially retain the upper cam 22 on the fastening rod 44.

The upper fastening element 26, or pin, and the fastening rod 44 thus define a first transverse fastening axis A1. The first fastening axis A1 is substantially parallel to the general pivoting axis of the upper lever 14.

It is noted that the radii of curvature r1, r2, r3 are not centered here on the first fastening axis A1.

Advantageously, the plate 24 is provided to be attached to the lower lever 16 in the same way as the upper cam 22 is provided to be attached to the upper lever 14. Also, the plate 24 is connected to a lower fastening element 50 forming a fastening pin provided with a threaded end section 28 intended to be screwed into a lower hole 52 made beforehand in the lower lever 16.

The lower fastening element 50 also comprises a fastening rod 44 provided with a splined section 46 enabling the plate 24 to be fastened.

The plate 24 comprises a base 54, or main body, which widens upward forming an upper track 56 able to receive the cam surface 32 bearing vertically (V) thereupon.

The upper track 56 is a flat surface with a rectilinear profile in the sagittal plane P1 and in a transverse plane. The upper track 56 generally follows the orientation of the lower support surface 20 when the orthopedic device 10 is mounted on the joint 12. According to the example shown in the figures, the upper track 56 is slightly inclined forward, that is, sloped forward.

The base 54 comprises at its lower end a splined hub element 42 which allows the plate 24 to be fitted onto the splined section 46 of the associated fastening rod 44.

The lower fastening element 50, or pin, and the associated fastening rod 44 thus define a second transverse fastening axis A2, substantially parallel to the first fastening axis A1.

Advantageously, the upper track 56 extends tangentially relative to the associated fastening rod 44 and it is offset rearward relative to a transverse plane passing through the associated fastening rod 44 when the orthopedic device 10 is mounted on the joint 12.

The upper track 56 can be made directly on the upper face of the base 54. It is also possible to provide one or more shims 58 capable of being removably attached to the plate 24.

FIG. 1 shows an exemplary embodiment wherein the orthopedic device 10 is provided with two shims 58 of different thicknesses, which can be removably attached to the upper face of the plate 24. These shims 58 make it possible, after drilling the holes 30, 52 in the levers 14, 16 and attaching the plate 24 to the lower lever 16, to selectively modify the radial distance, or center distance Dx, between the two fastening axes A1, A2. For example, they make it possible to correct a positioning too far from the fastening axes A1, A2 due for example to fluctuations during drilling operations in the levers 14, 16.

The shims 58 can also be used to slightly increase the center distance Dx so as to relieve the joint 12 by forcing the unloading of the forces toward the orthopedic device 10, that is to say towards the bearing of the upper cam 22 on the plate 24.

Here, each shim 58 is in the form of a plate able to completely cover the upper track 56 on the base 54. Each shim 58 can be attached for example by screwing in the base 54, at each longitudinal end.

It will be noted that the number of splines on each splined section 46 and on each splined hub element 42 determines the number of steps for the relative angular positioning of the upper cam 22 and of the plate 24 on the associated fastening rod 44.

According to the teachings of the invention, the profile of the cam surface 32 is shaped to ensure continuous contact of the upper cam 22 against the upper track 56, during the entire pivoting of the upper lever 14 relative to the lower lever 16, when the orthopedic device 10 is mounted on the joint 12. This specific shape is associated with a precise relative positioning of the cam surface 32 relative to the upper track 56, in particular defined by the center distance Dx between the fastening axes A1, A2.

Advantageously, the profile of the cam surface 32 is shaped so that, when the orthopedic device 10 is mounted on the joint 12, the upper cam 22 is pressed against the upper track 56:
by its front section 34, in the extended position, as shown in FIG. 2,
by its intermediate section 36, in the intermediate positions shown in FIGS. 3 and 4,
by its rear section, in the flexing position, as shown in FIG. 5.

As shown in FIGS. 2 to 5, during the pivoting of the upper lever 14 toward its flexing position, the first fastening axis A1 moves first from the front to the rear and then from the rear to the front, so that the position of the first fastening axis A1, when the joint 12 is in the flexing position, is shifted forward relative to its position when the joint 12 is in the extended position. The profile of the cam surface 32 is therefore also shaped to follow this movement of the first fastening axis A1 by allowing the upper cam 22 to slide on the upper track 56 of the plate 24.

An advantage of the orthopedic device 10 according to the invention is that it is configured to allow this sliding of the upper cam 22 on the upper track 56. It thus makes it possible to follow as closely as possible the kinematics of the movement of the joint 12, with its imperfections, variations, including angular movements, in a horizontal transverse plane, of the upper lever 14 relative to the lower lever 16. The particular shape of the cam surface 32 and the planar shape of the upper track 56 make it possible to compensate for the variations in relative positions of the upper lever 14 relative to the lower lever 16 and thus to adapt more easily to the specificities of each joint 12.

It should be noted that the width of the upper track 56, that is, its transverse dimension, is preferably greater than the width of the cam surface 32 so as to compensate for variations in transverse position of the upper cam 22 related to a transverse deflection of the upper lever 14 relative to the lower lever 16, during pivoting.

A method for mounting the orthopedic device 10 according to the invention on the joint 12 is now described, starting from the extended position of the joint 12.

According to a first step, the appropriate location is determined in the levers 14, 16 for attaching the fastening elements 26, 50 thereto, taking into account the kinematics of the joint 12 and the levers 14, 16 during pivoting from the extended position to the flexing position. This step advantageously comprises a modeling of the joint 12 so as to identify the best location for producing the holes intended for the fastening of the levers 14, 16.

A drilling step is then carried out so as to produce the upper hole 30 in the upper lever 14 and the lower hole 52 in the lower lever 16. Here, the objective is to ensure the best possible parallelism between the two fastening axes A1, A2, and to obtain a center distance Dx of predetermined value.

During a subsequent step, the fastening elements 26, 50, or pins, with the fastening rods 44, are screwed into their respective holes 30, 52.

During a next step, the plate 24 is sleeved onto the splined section 46 of the lower fastening rod 44, by choosing the angular position on the fastening rod 44 as a function of the desired inclination of the upper track 56, following the general inclination of the upper bearing surface 18, or tibial plate.

Once the plate 24 is fitted onto the splined section 46 in the correct angular position, the plate 24 is held in position by screwing a locking screw 48.

During another step, the upper cam 22 is fitted onto the splined section 46 of the upper fastening rod 44, by choosing the angular position on the fastening rod 44 so that the cam surface 32 bears on the upper track 56 by its front section 34.

It should be noted that the larger the number of splines on the splined section 46, the more precise the adjustment of the angular position of the upper cam 22 on the associated fastening rod 44.

The upper cam 22 is then held on the fastening rod 44 by means of a locking screw 48.

According to the embodiment described above, the upper cam 22, and more particularly the profile of the cam surface 32, is specifically designed for a given patient, so as to precisely adapt the cam surface 32 to the kinematics of the joint 12 of the patient.

According to one alternative embodiment, a series of several upper cams 22 is provided, each with a different profile for the cam surface 32, so as to be able to adapt to a corresponding series of types of joints 12. It is then possible, during the step of modeling the joint 12, to determine which model of upper cam 22 is most suitable for the joint of a specific patient. This makes it possible not to have to design a specific upper cam 22 for each patient.

A range of upper cams 22, with associated plates 24, with different sizes and profiles, can be created from statistical data obtained during the production of a specific number of upper cams 22 and plates 24 customized per patient.

One advantage of the present invention is that it is possible to produce a single type of plate 24 capable of adapting to different upper cams 22 for different joints 12.

According to an alternative embodiment, it is possible to provide angular markers on the base 54 to facilitate the angular positioning of the plate 24 on its fastening rod 44 and thus define the slope of the upper track 56.

By virtue of the invention, it is possible to design a prosthetic device 10 comprising parts identical from one patient to another, in particular the fastening elements 26, 50 and the fastening rods 44, which reduces manufacturing costs.

According to an advantageous embodiment, the upper cam 22 and/or the plate 24 can be produced by an additive manufacturing method, depending on the parameters of the joint 12 of the selected patient. The upper cam 22 and the plate 24 are for example made of titanium.

It should be noted that the orthopedic device 10 according to the invention, which has been described above for a knee joint, could also be applied to another type of joint, for example to the joint of an ankle.

An advantage of the orthopedic device 10 according to the invention is that the external mechanism, that is, the assembly formed by the upper cam 22 and the plate 54, can be removed, or exchanged, or repaired, or adapted at any time of the therapy, without surgical intervention, which allows unequalled agility in the support of the pathologies associated with the joint 12.

### LEGEND

10: orthopedic device
12: joint
14: upper lever
16: lower lever
18: upper bearing surface
20: lower bearing surface
22: upper cam
24: plate
26: upper fastening element
28: threaded end section
30: upper hole
32: cam surface
34: front section
36: intermediate section
38: rear section
40: main body
42: splined hub element
44: fastening rod
46: splined section
48: locking screws
50: lower fastening element
52: lower hole
54: base
56: upper track
58: shim
a1: flexing angle
A1: first fastening axis
A2: second fastening axis
Dx: center distance
P1: sagittal plane
r1: first radius of curvature
r2: second radius of curvature
r3: third radius of curvature

1. An orthopedic device intended to equip a joint, said joint comprising an upper lever and a lower lever, the respective ends of which have an upper bearing surface and a lower bearing surface opposite one another, said levers being pivotable relative to one another around their respective bearing surfaces and in a pivoting plane called sagittal plane, between an extension position wherein said levers form between them an angle of at most 180 degrees, and a flexing position wherein said levers form between them an angle of at least 20 degrees, the orthopedic device comprising an upper cam and a plate not directly connected to one another, the upper cam being joined to an upper fastening element provided to rigidly connect the upper cam to the upper lever, the plate being joined to a lower fastening element provided to rigidly connect the plate to the lower lever, the upper cam and the plate being provided in order to be arranged outside the joint so as to relieve the joint when it is under load,
wherein the upper cam has a cam surface whose generatrix has a convex curved profile, in the sagittal plane,
in that the plate comprises a substantially planar upper track, with a rectilinear profile in the sagittal plane,
the profile of the cam surface and the relative positioning of the cam surface with respect to the upper track being configured to ensure continuous contact of the upper cam against the upper track during the entire pivoting of the upper lever relative to the lower lever, when the orthopedic device is mounted on the joint, by making it possible to immediately follow the kinematics of the movement of the joint, with its imperfections, variations, including angular movements, in a horizontal transverse plane, of the upper lever relative to the lower lever,
and in that the plate and/or the upper cam are each connected to the associated fastening element by a single fastening rod.

2. The orthopedic device according to claim 1, wherein each fastening element consists of a pin which is provided to be attached in a portion of the associated lever of the joint, and in that the pin extends on the opposite side of the lever to form the fastening rod.

3. The orthopedic device according to claim 1, wherein the fastening rod comprises a splined section and in that the upper cam and the plate are each provided with a splined hub element complementary to the associated splined section in order to allow the upper cam and the plate to be attached respectively to the associated fastening rod, the number of splines on the splined section determining the number of steps for the relative angular positioning of the upper cam and of the plate on the associated splined section.

4. The orthopedic device according to claim 1, wherein the upper track is offset towards the rear relative to a vertical transverse plane passing through the associated fastening rod, when the orthopedic device is mounted on the joint.

5. The orthopedic device according to claim 1, wherein the profile of the cam surface is configured so that the upper cam is in abutment against the upper track by a front section of the cam surface, when the orthopedic device is mounted on the joint and in the extended position, and so that the upper cam is bearing against the upper track by a rear section of the cam surface, when the orthopedic device is mounted on the joint and in the flexing position.

6. The orthopedic device according to claim 1, wherein the cam surface has several radii of curvature along its profile in the sagittal plane.

7. The orthopedic device according to claim 1, wherein the main body forming the upper cam is of increasing transverse thickness from the fastening rod toward the cam surface.

8. The orthopedic device according to claim 1, wherein the upper cam is made in a single piece with the splined hub element.

9. The orthopedic device according to claim 1, wherein the upper track is arranged on a shim that is removably attached to the plate.

10. The orthopedic device according to claim 1, wherein it is configured to equip a knee joint, the upper lever corresponding to a femur and the lower lever corresponding to a tibia.
